(19) United States
(12) Patent Application Publication
(10) Pub. No.: US 2021/0030785 A1
(43) Pub. Date: Feb. 4, 2021

(54) METHOD OF PROPHYLAXIS OF ZIKA VIRUS INFECTION

(71) Applicant: Starpharma Pty Ltd., Victoria (AU)

(72) Inventors: Jeremy Robert Arthur PAULL, Victoria (AU); Jessica HUBERMAN, Victoria (AU)

(21) Appl. No.: 16/098,783

(22) PCT Filed: May 4, 2017
(86) PCT No.: PCT/AU2017/050408

§ 371 Date: Nov. 2, 2018

(30) Foreign Application Priority Data
May 5, 2016 (AU) 2016901650

(51) Int. Cl. A61K 31/795; A61K 9/00

(52) U.S. Cl. CPC A61K 31/795; A61K 9/0014; A61K 9/0034

(57) ABSTRACT

Provided herein are methods for preventing transmission of Zika virus, and of preventing diseases, disorders and symptoms associated with Zika virus infections, in particular for preventing transmission during sexual intercourse. The methods comprise topical administration of a macromolecule comprising a dendrimer of 1 to 8 generations with one or more sulfonic acid- or sulfonate-containing moieties attached to one or more surface groups of the dendrimer. Also provided herein are related uses, compositions, devices and systems.

### FIELD OF THE INVENTION

The present invention relates to methods of preventing Zika virus infections.

Methods, devices and compositions for preventing the spread of Zika virus by sexual transmission are also described.

### BACKGROUND OF THE INVENTION

Zika virus is a member of the virus family Flaviviridae and the genus Flavivirus, and is related to dengue, yellow fever, Japanese encephalitis, and West Nile viruses. Zika virus is an enveloped, icosahedral, single-stranded, positive-sense RNA virus.

Zika virus disease, caused by the Zika virus, is spread to people primarily through the bite of an infected Aedes species mosquito. The illness is usually mild with symptoms lasting for several days to a week after being bitten by an infected mosquito. In some cases, Zika infection can cause fever, rash, severe headache, joint pain, and muscle or bone pain. However, Zika virus infection during pregnancy can cause a serious birth defect called microcephaly, as well as other severe fetal brain defects. Because people symptoms are generally mild, many people do not realize they have been infected.

While the primary route of transmission is via mosquito, transmission via other routes is also documented. Authorities warn that Zika may be transmitted by vaginal, anal or oral sex, and the virus is detectable in semen for longer than plasma. As a result, Health Authorities recommend using a condom or abstaining from sex if infected and for at least 6 months thereafter. However, condoms have been known to rupture due to stresses, caused by, for example, stretching or incorrect use. Condoms may also develop microscopic leaks, or may contain small perforations that may lead to transfer of bodily fluids across the barrier, leading to risk of infection. Mild symptoms are usually treated with nonsteroidal anti-inflammatory drugs.

No vaccine or other medication exists to prevent Zika virus infection attained by any route of transmission. Effective vaccines exist for several viruses of the flaviviridae family, e.g. Yellow fever vaccine, Japanese encephalitis vaccine, and Tick-borne encephalitis vaccine. However, vaccines are virus specific. A number of companies are working towards developing a vaccine for Zika but none had yet reached clinical trials, and many years may be needed before an effective Zika vaccine is approved for public use.

Accordingly, there remains a need for agents capable of preventing or reducing the spread of Zika virus, and of associated conditions.

Antiviral dendrimcrs have been developed with activity against HIV, HPV and HSV, see for example WO02/079299 and WO2007/045009. However, antiviral agents are generally selective in their action against viruses. There are no approved broad spectrum antiviral agents for broad classes of viral agents such as RNA virus. Even within a family, such as Herpes viridae, agents effective against one virus are not usually effective for others, e.g. treatments against varicella, EBV or HSV are not mutually effective. Efforts to find a broad spectrum antiviral have generally been targeted to enhancing the immune response non-specifically against intracellular pathogens.

### SUMMARY OF THE INVENTION

The present inventors have found that the dendrimcric macromolecule SPL7013 has prophylactic activity against the Zika virus in vitro. Accordingly it is envisaged that SPL7013 and structurally-related compounds will find utility in reducing the transmission of Zika virus, and in preventing or reducing the incidence of associated conditions. This is unexpected in view of experimental data which indicates that dendrimeric compounds are inactive against related viruses such as Dengue fever virus and West Nile fever virus.

Accordingly, in a first aspect there is provided a method of preventing or reducing the likelihood of transmission of Zika virus during sexual intercourse to a first individual from a second individual infected with Zika virus, comprising:

topically administering to the first and/or second individual an effective amount of
a macromolecule or a pharmaceutically acceptable salt thereof, or
a composition comprising the macromolecule or pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer.

In another aspect there is provided a method of preventing or reducing the likelihood of a disease, disorder or symptom associated with a Zika virus infection in a first individual, by preventing transmission of Zika virus during sexual intercourse to the first individual from a second individual infected with Zika virus, the method comprising:

topically administering to the first and/or second individual an effective amount of
a macromolecule or a pharmaceutically acceptable salt thereof, or
a composition comprising the macromolecule or pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer.

In another aspect there is provided a method of preventing or reducing the likelihood of transmission of Zika virus during sexual intercourse to a first individual from a second individual infected with Zika virus, the method comprising: using during sexual intercourse of a system comprising:
a) a prophylactic device; and
b) a composition comprising an effective amount of a macromolecule or a pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer.

In another aspect there is provided a method of preventing or reducing the likelihood of transmission of Zika virus to an individual, comprising:

topically administering to the individual an effective amount of
a macromolecule or a pharmaceutically acceptable salt thereof, or
a composition comprising the macromolecule or pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer.

In another aspect there is provided a method of preventing or reducing the likelihood of a disease, disorder or symptom associated with a Zika virus infection in an individual comprising:

topically administering to the individual an effective amount of
a macromolecule or a pharmaceutically acceptable salt thereof, or
a composition comprising the macromolecule or pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer.

In another aspect there is provided a macromolecule or a pharmaceutically acceptable salt thereof,

or a composition comprising the macromolecule or pharmaceutically acceptable salt thereof and a pharmaceutically acceptable carrier,

for use in a method of preventing or reducing the likelihood of transmission of Zika virus during sexual intercourse to a first individual from a second individual infected with Zika virus, the method comprising:

topically administering to the first and/or second individual an effective amount of the macromolecule or pharmaceutically acceptable salt thereof, or of the composition,

wherein the macromolecule comprises a dendrimer of 1 to 8 generations with one or more sulfonic acid- or sulfonate-containing moieties attached to one or more surface groups of the dendrimer.

In another aspect there is provided a macromolecule or a pharmaceutically acceptable salt thereof,

or a composition comprising the macromolecule or pharmaceutically acceptable salt thereof and a pharmaceutically acceptable carrier,

for use in a method of preventing or reducing the likelihood of a disease, disorder or symptom associated with a Zika virus infection in a first individual, by preventing transmission of Zika virus during sexual intercourse to the first individual from a second individual infected with Zika virus, the method comprising:

topically administering to the first and/or second individual an effective amount of the macromolecule or pharmaceutically acceptable salt thereof, or of the composition,

wherein the macromolecule comprises a dendrimer of 1 to 8 generations with one or more sulfonic acid- or sulfonate-containing moieties attached to one or more surface groups of the dendrimer.

In another aspect there is provided a macromolecule or a pharmaceutically acceptable salt thereof,

or a composition comprising the macromolecule or pharmaceutically acceptable salt thereof and a pharmaceutically acceptable carrier,

for use in preventing or reducing the likelihood of transmission of Zika virus to an individual,

wherein the macromolecule comprises a dendrimer of 1 to 8 generations with one or more sulfonic acid- or sulfonate-containing moieties attached to one or more surface groups of the dendrimer.

In another aspect there is provided a macromolecule or a pharmaceutically acceptable salt thereof,

or a composition comprising the macromolecule or pharmaceutically acceptable salt thereof and a pharmaceutically acceptable carrier,

for use in preventing or reducing the likelihood of a disease, disorder or symptom associated with a Zika virus infection,

wherein the macromolecule comprises a dendrimer of 1 to 8 generations with one or more sulfonic acid- or sulfonate-containing moieties attached to one or more surface groups of the dendrimer.

In another aspect there is provided a system comprising:
a) a prophylactic device; and
b) a composition comprising an effective amount of a macromolecule or a pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer,

for use in preventing or reducing the likelihood of transmission of Zika virus to an individual.

In another aspect there is provided a system comprising:
a) a prophylactic device; and
b) a composition comprising an effective amount of a macromolecule or a pharmaceutically acceptable salt thereof and a pharmaceutically acceptable carrier,

wherein the macromolecule comprises a dendrimer of 1 to 8 generations with one or more sulfonic acid- or sulfonate-containing moieties attached to one or more surface groups of the dendrimer,

for use in preventing or reducing the likelihood of a disease, disorder or symptom associated with a Zika virus infection.

In another aspect there is provided use of a macromolecule or a pharmaceutically acceptable salt thereof, for the manufacture of a medicament for the prevention or reduction of likelihood of transmission of Zika virus to an individual, wherein the macromolecule comprises a dendrimer of 1 to 8 generations with one or more sulfonic acid- or sulfonate-containing moieties attached to one or more surface groups of the dendrimer

In another aspect there is provided use of a macromolecule or a pharmaceutically acceptable salt thereof, for the manufacture of a medicament for the prevention or reduction of likelihood of a disease, disorder or symptom associated with a Zika virus infection, wherein the macromolecule comprises a dendrimer of 1 to 8 generations with one or more sulfonic acid- or sulfonate-containing moieties attached to one or more surface groups of the dendrimer.

In some embodiments, the macromolecule or pharmaceutically acceptable salt thereof is a dendrimer comprising lysine building units of from 3 to 6 generations, and the sulfonic acid- or sulfonate-containing moieties are napthyldisulfonate moieties. In some embodiments, the macromolecule is SPL7013.

In some embodiments, the macromolecule is used in the form of a composition which is a lubricant composition for sexual intercourse.

In some embodiments of the methods of the present disclosure, the method comprises using during sexual intercourse a system comprising:
a) a prophylactic device; and
b) a composition comprising the macromolecule or a pharmaceutically acceptable salt thereof and a pharmaceutically acceptable carrier.

In some embodiments, the composition is applied to the prophylactic device. In some embodiments, the system is a condom coated with a composition comprising the macromolecule or a pharmaceutically acceptable salt thereof and the pharmaceutically acceptable carrier.

In some embodiments, the disease disorder or symptom is Zika virus disease.

In some embodiments, the individual or first individual is a female individual.

### DESCRIPTION OF THE INVENTION

### Definitions

The articles “a” and “an” are used herein to refer to one or to more than one (i.e. to at least one) of the grammatical object of the article. By way of example, “an element” means one element or more than one element.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “about” refers to a quantity, level, value, dimension, size, or amount that varies by as much as 30%, 25%, 20%, 15%, 10%, 5% or 1% to a reference quantity, level, value, dimension, size, or amount.

As used herein, the term “individual” refers to any individual susceptible to Zika virus infection. In a particular embodiment, the individual is a human, including fetus, child and adult. In some embodiments the individual is a human adult. In some embodiments the individual is pregnant.

The term “prevention” or “prophylaxis” refers to reducing the risk of contracting or developing infection. Prevention need not be complete and does not imply that a subject will not eventually contract or develop the infection.

### Macromolecules

The present disclosure involves the use of macromolecules and/or pharmaceutically acceptable salts thereof. The pharmaceutically acceptable salt may be of any suitable type. Examples of suitable salts include, but are not limited to metallic salts (for example, aluminium, calcium, lithium, magnesium, potassium, sodium and zinc salts), organic salts (for example, organic amines such as N,NI-dibenzylethylenediamine, chloroprocaine, diethanolamine, ethylenediamine, dicyclohexylamine, cyclohexylamine, meglumine, (N-methylglucamine) and procaine), quaternary amines (for example, choline), sulphonium salts and phosphonium salts. In particular embodiments, salts are selected from sodium and potassium, especially sodium.

Those skilled in the art will appreciate that many organic compounds can form complexes in solvents in which they are reacted or from which they are precipitated or crystallized. These complexes are known as “solvates”. For example, a complex with water is known as a “hydrate”. Solvates, such as hydrates, exist when the compound incorporates solvent. It will be understood that the macromolecules of the present invention, as well as salts thereof, may be present in the form of solvates. Solvates of the macromolecules which are suitable are those where the associated solvent is pharmaceutically acceptable.

The macromolecules used in the present invention comprise dendrimers of 1 to 8 generations with one or more sulfonic acid- or sulfonate-containing moieties attached to one or more surface groups of the dendrimer. The dendrimers useful in the invention may be any suitable dendrimer of 1 to 8 generations that is capable of presenting one or more sulfonic acid- or sulfonate-containing moieties on its surface. In some embodiments, the dendrimer is selected from a polylysine, polyglutamate, polyaspartate, polyamidoamine (PAMAM), poly(etherhydroxylamine), polyether, polyester or poly(propyleneimide) (PPI) dendrimer having 1 to 8 generations. The macromolecule also comprises one or more sulfonic acid- or sulfonate-containing moieties attached to the one or more surface functional groups of the outermost generation of the dendrimer. For example, when the dendrimer is a polylysinc, polyamidoamine, poly(etherhydroxylamine) or poly(propyleneimide) dendrimer, the surface functional groups are amino groups, and when the dendrimer is a polyglutamate or polyaspartate dendrimer, the surface functional groups are carboxylic acids.

Dendrimers are branched polymeric macromolecules composed of multiple branched monomers radiating from a central core moiety. The number of branch points increases upon moving from the dendrimer core to its surface and is defined by successive layers or “generations” of monomers (or building units). Each generation of building units is numbered to indicate the distance from the core. For example, Generation 1 (GI) is the layer of building units attached to the core, Generation 2 (G2) is the layer of building units attached to Generation 1, Generation 3 (G3) is the layer of building units attached to Generation 2, and so on.

The outermost generation of building units provides the surface of the dendrimer and presents functional groups, to which the at least one sulfonic acid- or sulfonate-containing moiety is covalently bonded. The sulfonic acid- or sulfonate-containing group may be directly bonded to the surface functional group or may be attached to the surface functional group through a linker.

The dendrimers contemplated herein can be prepared by methods known in the art. For example, they may be prepared in either a convergent manner (where, effectively, the branches are pre-formed and then attached to the core) or a divergent manner (where the layers or generations are successively built outwards from the core). Both these methods would be well understood to the skilled person.

The dendrimers may comprise any suitable core. As used herein, “core” refers to the moiety upon which generations of monomers or building units are built (either through a divergent process or a convergent process), and may be any moiety having at least one reactive or functional site from which layers of monomer or building units are successively generated (or to which a pre-formed “branch” is attached). Some exemplary suitable cores contemplated herein include those having 1, 2, 3 or 4 reactive groups independently selected from, amino, carboxyl, thiol, alkyl, alkynyl, nitrile, halo, azido, hydroxylamine, carbonyl, maleimide, acrylate or hydroxy groups to which the layers or generations of building units or monomers can be attached. A core moiety may be the same as a building unit or may be different.

Exemplary cores include polyaminohydrocarbons, disulfide containing polyamines, poly(glycidyl ethers), aminoethanol, ammonia, arylmethylhalides, piperazine, aminoethylpiperazine, poly(ethyleneimine), alkylene/arylene dithiols, 4,4-dithiobutyric acid, mercaptoalkylamines, thioether alkylamines, isocyanurate, heterocycles, macrocycles, polyglycidylmethacrylate, phosphine, porphines, oxiranes, thiorancs, oxetanes, aziridines, azetidines, multiazidofunctionalities, siloxanes, oxazolines, carbamates or caprolactones.

Some non-limiting examples of core moieties contemplated herein include ammonia and diamino C₂-C₁₂ alkanes such as ethylene diamine, 1,4-diaminobutane and 1,6-diaminohexane. However, it will be appreciated that the core is not necessarily a linear moiety with a single reactive group at each end. Non-linear, cyclic or branched core moieties are also contemplated by the present invention. For example, arylmethylamines such as benzhydrylamine (BHA), are suitable cores. In some embodiments, the core is a benzhydrylamine (BHA) core:

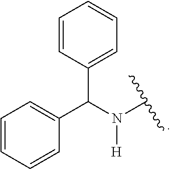 [SMILES: *NC(c1ccccc1)c1ccccc1]

The dendrimers also comprise one or more building units. In some embodiments, the building units of the dendrimer are selected from:

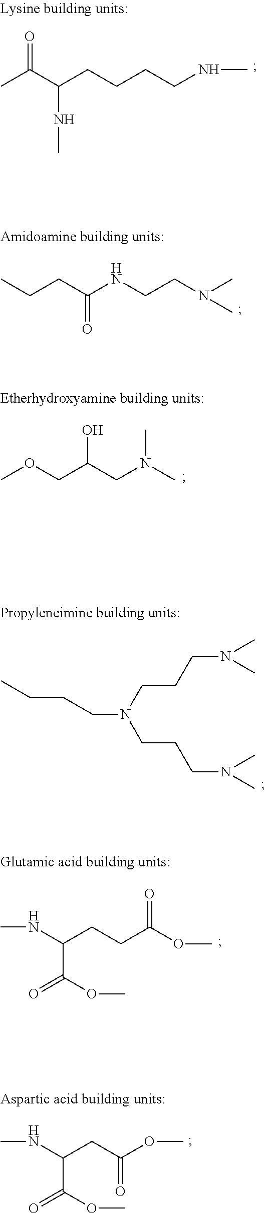

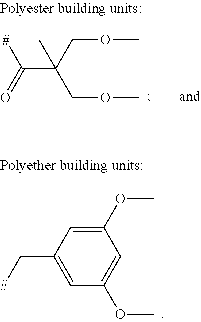

In some embodiments, the building unit or building units of the dendrimer are lysine or lysine analogues selected from a compound of the following formula:

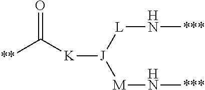

wherein K is absent or is selected from —C₁₋₆ alkylene-, —C₁₋₆ alkyleneNHC(O)—, —C₁₋₆ alkyleneC(O)—, -C₁₋₃ alkylene-O—C₁₋₃alkylene-, —C₁₋₃ alkylene-O—C₁₋₃ alkyleneNHC(O)— and —C₁₋₃ alkylene-O—C₁₋₃ alkyleneC(O)—;
J is selected from CH or N;
L and M are independently absent or is selected from —C₁₋₆alkylene- or —C₁₋₃ alkyleneOC₁₋₃alkylene; provided that when L and/or M are absent, J is CH;
** indicates the linkage between the lysine or lysine analogue and the core of the dendrimer or the previous generation of building units; and
*** indicates the linkage between the lysine or lysine analogue and the subsequent generation of lysine or lysine analogues or forms the surface amino groups of the dendrimer.

Exemplary lysine analogue building units including the following:
Glycyl-Lysine 1 having the structure:

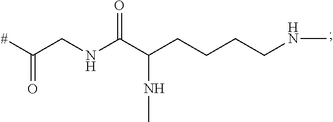

Analogue 2, having the structure below, where a is an integer 1 or 2; and b and c are independently integers 1, 2, 3 or 4:

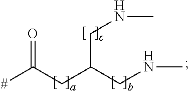

Analogue 3, having the structure below, where a is an integer 0, 1 or 2; and b and c are independently integers 2, 3, 4, 5 or 6:

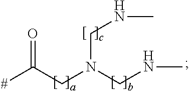

and
Analogue 4, having the structure below, where a is an integer 0, 1, 2, 3, 4 or 5; and b and c are independently integers 1, 2, 3, 4 or 5:

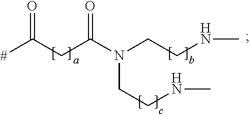

wherein each # denotes the carbonyl residue of the carboxyl group which forms an amide bond with a nitrogen atom of the core or a nitrogen atom of a previous generation of building units;
and wherein any methylene group of the building units may be replaced by a mcthylencoxy (CH₂O) or ethylencoxy (CH₂—CH₂—O) group, provided that this does not result in the formation of a carbonate (—O—C(O)—O—) or carbamate (—O—C(O)—N—) moiety within the building unit.

Other suitable building units include:

Analogue 5, having the structure below, where a is an integer of 0 to 2; b and c are the same or different and are integers of 1 to 4; Aₗ and A, are the same or different and selected from NH₂, CO₂H, OH, SH, X, Allyl-X, epoxide, aziridine, N₃ or alkyne, where X is F, Cl, Br or I,

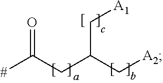

Analogue 6, having the structure below, where a is an integer of 0 to 2; b and c are the same or different and are integers of 2 to 6; A₁ and A₂ are the same or different and selected from NH,, CO₂H, OH, SH, X, Allyl-X, epoxide, aziridine, N₃ or alkyne, where X is F, Cl, Br or I,

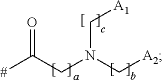

and
Analogue 7, having the structure below, where a is an integer of 0 to 5; b and c are the same or different and are integers of 1 to 5; A₁ and A₂ are the same or different and selected from NH₂, CO₂H, OH, SH, X, Allyl-X, epoxide, aziridine, N₃ or alkyne, where X is F, Cl, Br or I,

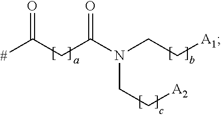

wherein each # denotes the carbonyl residue of the carboxyl group which forms an amide bond with a nitrogen atom of the core or a nitrogen atom of a previous generation of building units;
and wherein any methylene group of the building units may be replaced by a methyleneoxy (CH₂—O) or ethyleneoxy (CH₂—CH₂—O) group, provided that this does not result in the formation of a carbonate (—O—C(O)—O—) or carbamate (—O—C(O)—N—) moiety within the building unit.

In some embodiments, the macromolecule is a polylysine dendrimer having lysine building units, especially a polylysine dendrimer with a benzhydrylamine core, e.g. a dendrimer comprising a core and building units as shown below:

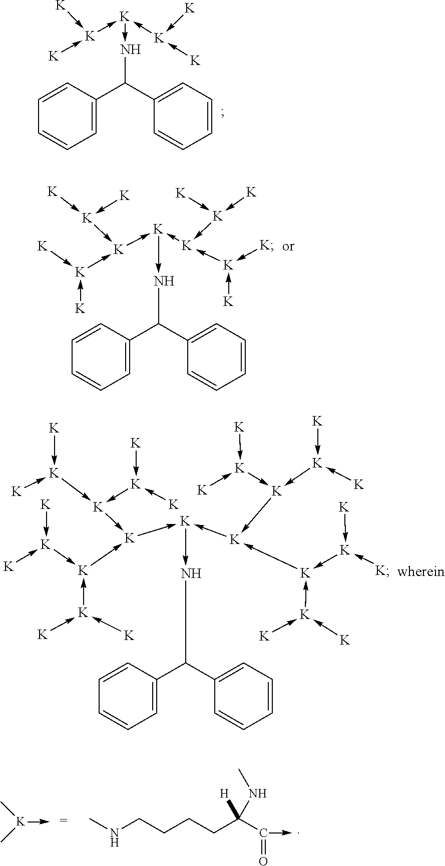

The sulfonic acid-containing or sulfonate-containing moiety is any moiety that is able to present the sulfonic acid or sulfonate group on the surface of the dendrimer. In some embodiments, the sulfonic acid- or sulfonate-containing moiety has one sulfonic acid or sulfonate group. In other embodiments, the sulfonic acid- or sulfonate-containing moiety has more than one sulfonic acid or sulfonate group, for example 2 or 3 sulfonic acid or sulfonate groups, especially 2 sulfonic acid or sulfonate groups. In some embodiments, the sulfonic acid- or sulfonate-containing moiety comprises an aryl group, such as a phenyl group or naphthyl group, especially a naphthyl group. In some embodiments, the sulfonic acid- or sulfonate-containing moiety comprises a naphthyl group substituted by two sulfonic acid or sulfonate moieties (also referred to as a napthyldisulfonate moiety), for example a 3,6-disulfonatonapthyl moiety.

When the sulfonate-containing moiety is present, the moiety may be present in ionic form (—SO₃⁻) or in the form of a salt, for example, the sodium salt (—SO₃Na).

Examples of suitable sulfonic acid or sulfonate-containing moieties include but are not limited to:
—NH—(CH₂)ₙSO₃⁻, —(CH₂)ₙSO₃⁻,

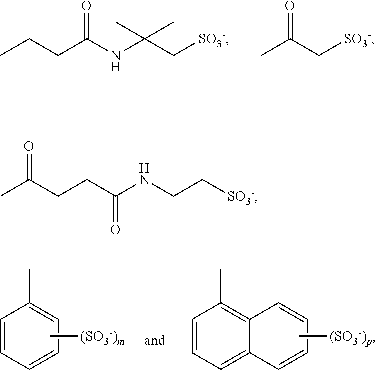

in which n is 0 or an integer of 1 to 20, m is an integer of 1 or 2 and p is an integer of 1 to 3.

In some embodiments the sulfonic acid- or sulfonate-containing moiety contains an aryl group, for example a phenyl group or a naphthyl group. In some embodiments, the sulfonic acid- or sulfonate-containing moiety is selected from:

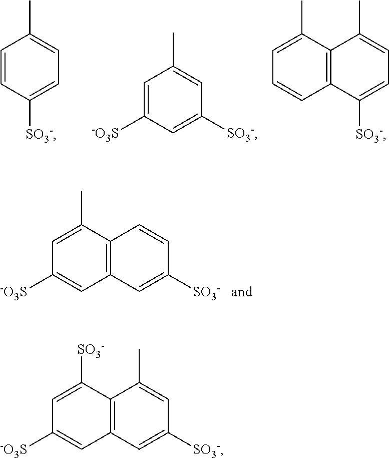

especially

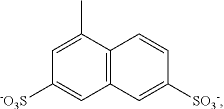

In some embodiments, the sulfonic acid- or sulfonate-containing moiety is directly bonded to the surface amino group of the dendrimer. In other embodiments, the sulfonic acid- or sulfonate-containing moiety is attached to the surface amino group of the dendrimer through a linker group.

Suitable linker groups include alkylene or alkenylene groups in which one or more non-adjacent carbon atoms is optionally replaced by an oxygen or sulfur atom to provide an ether, thioether, polyether or polythioether; or a group —X₁—(CH₂)_{q}—X, or —X—(CR₁R₂)_{q}—X— wherein X₁ and X₂ are independently selected from —NH—, —C(O)—, —O—, —S— and —C(S), R₁ and R, are independently selected from hydrogen or —CH₃, and q is 0 or an integer from 1 to 10, and in which one or more non-adjacent (CH₂) groups may be replaced with —O— or —S— to form an ether, thioether, polyether or polythioether.

In a particular embodiment, the linker is

#-O—CH₂—C(O)-*

in which # designates attachment to the sulfonic acid-containing moiety and * designates attachment to the surface amino group of the dendrimer.

In some embodiments the sulfonic acid- or sulfonate-containing moiety is attached to the surface amino group of the dendrimer through a linker group, and the linker-sulfonic acid/sulfonate moiety is:

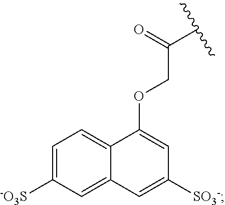

or a pharmaceutically acceptable salt thereof.

Exemplary dendrimers useful in the invention include those of formulae I, II and III:

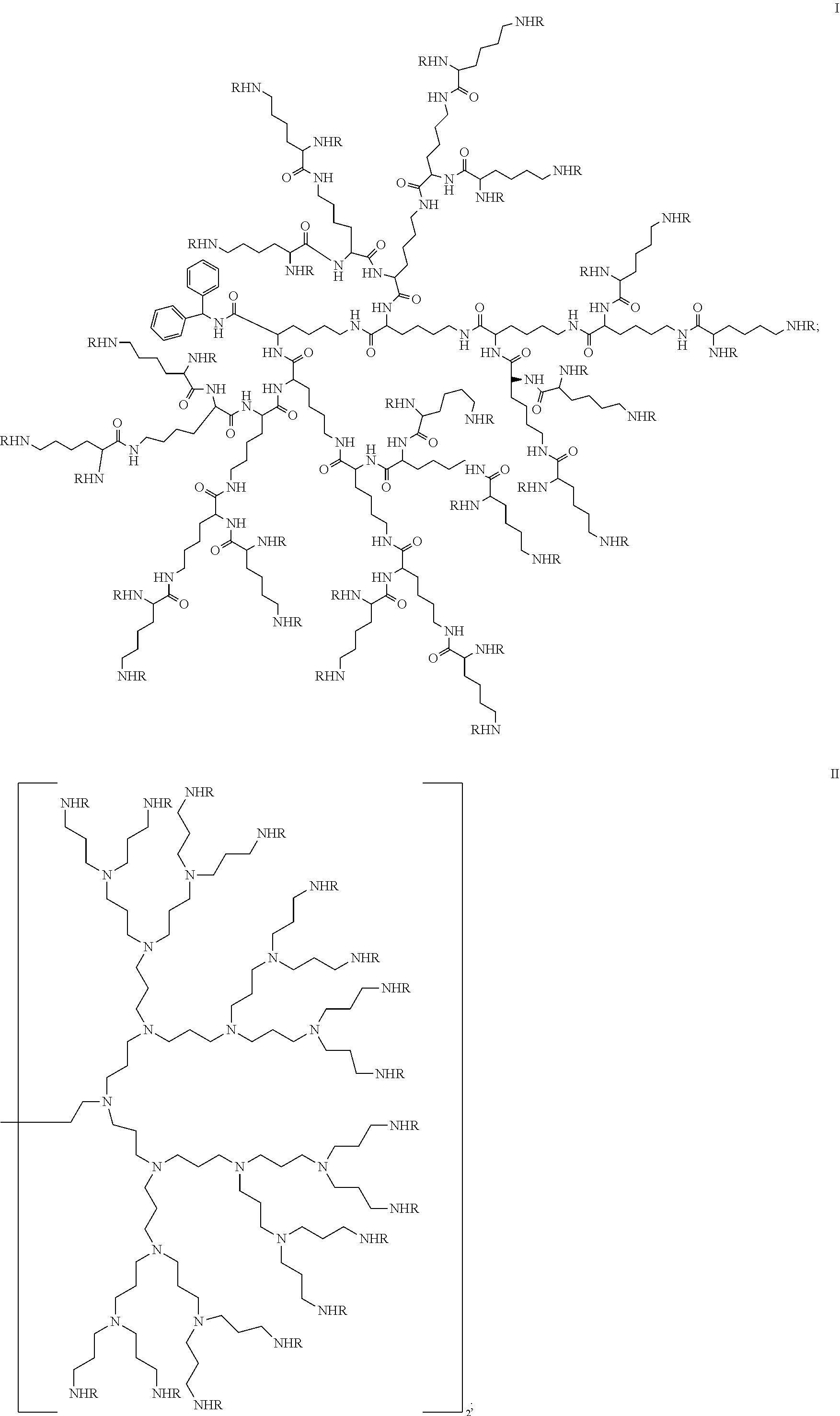

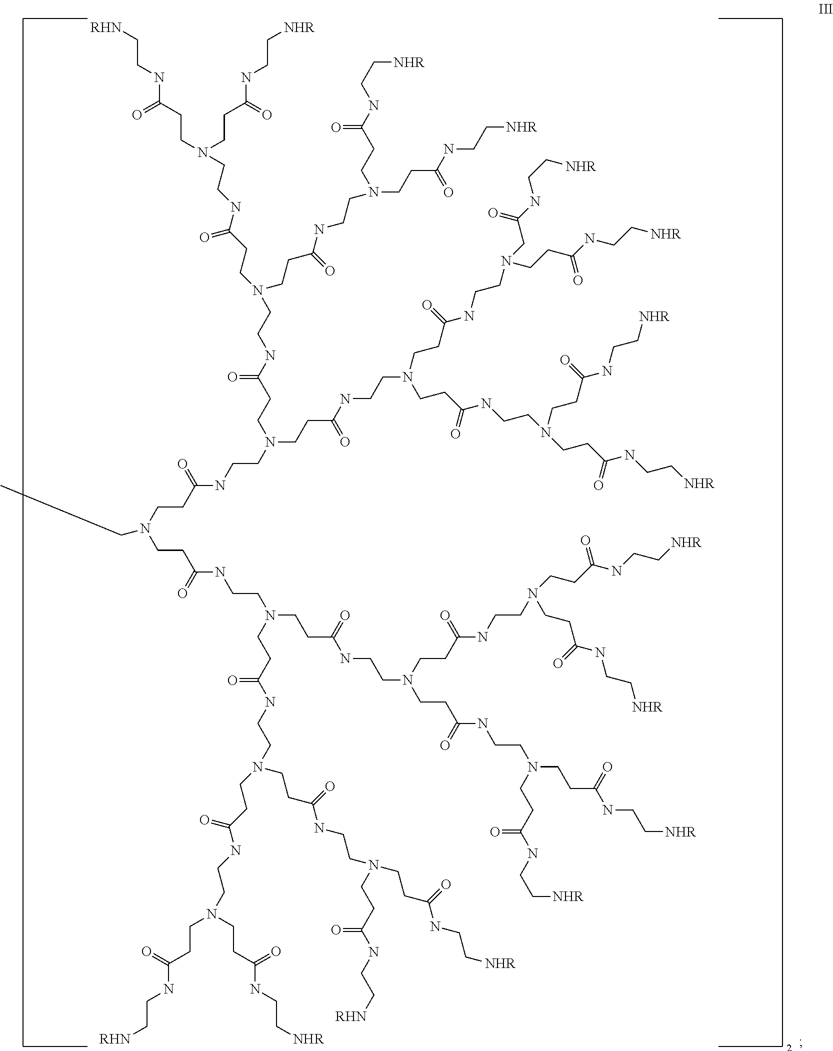

in which each R group is represented by a group formula IV or hydrogen:

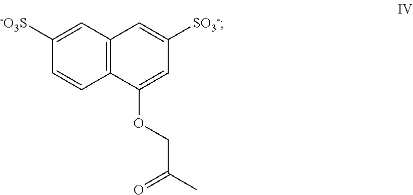

provided that at least one R group is a group of formula IV;
or a pharmaceutically acceptable salt thereof.

In particular embodiments, more than one R group is a group of formula IV, for example in some embodiments at least 10 of the R groups are groups of formula IV, at least 15 of the R groups are groups of formula IV, at least 20 of the R groups are groups of formula IV, at least 25 of the R groups are groups of formula IV or at least 30 of the R groups are groups of formula IV. In some embodiments, all of the R groups are groups of formula IV.

In some embodiments the macromolecule is a dendrimer of formula I:

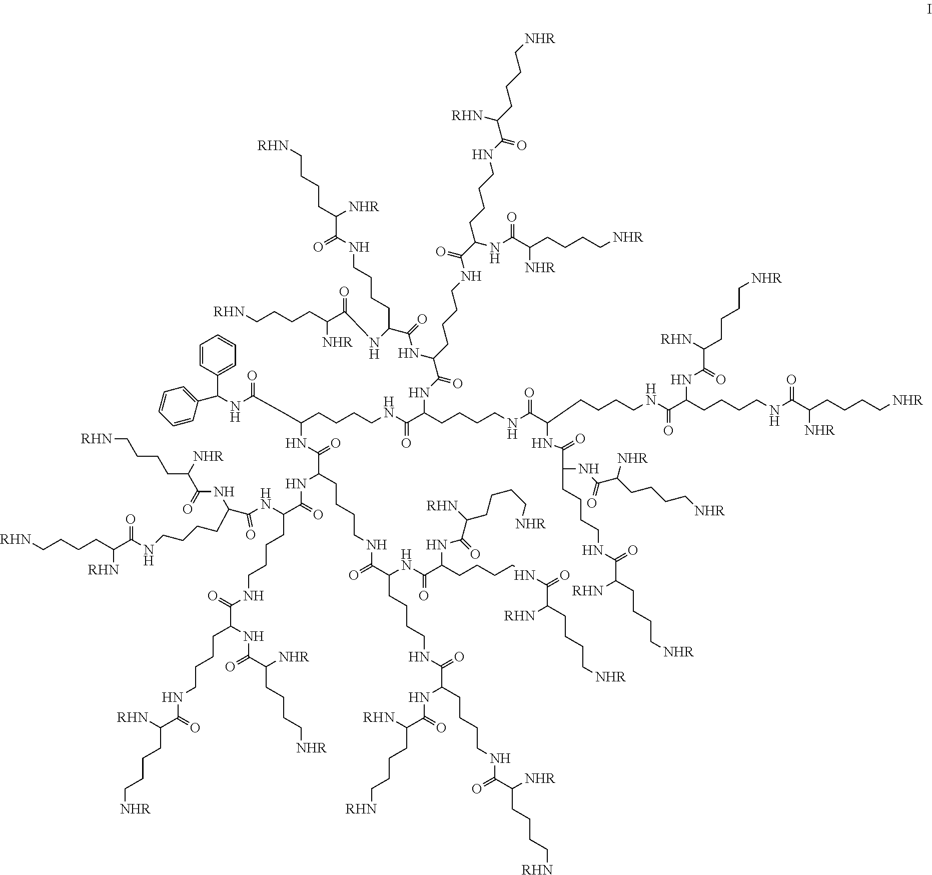

where R represents a group of the formula IV:

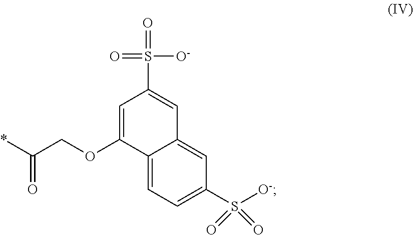

or a pharmaceutically acceptable salt of such a dendiimer.

A particular dendrimer of formula I has all R groups as groups of formula IV (SPL7013). SPL7013, also known as astodrimer sodium, has the structure:

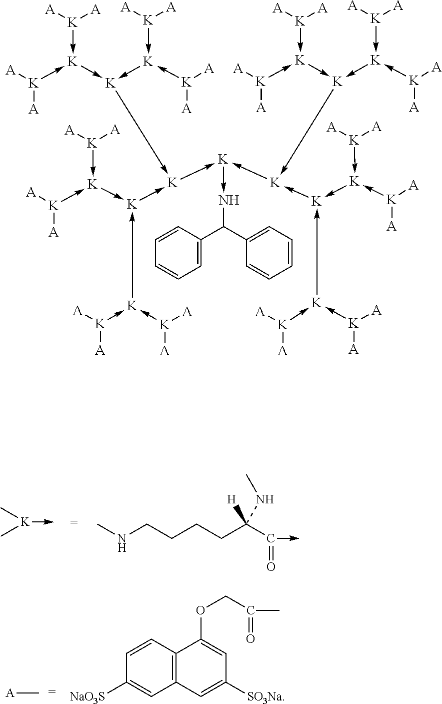

In some embodiments the macromolecule or pharmaceutically acceptable salt thereof is SPL7013 (astodrimer sodium).

A particular dendrimer of formula II has all R groups as groups of formula IV (SPL7320). A particular dendrimer of formula III has all R groups as groups of formula IV (SPL7304).

The synthesis of dendrimers of Formulae I, II and III is described in WO02/079299.

### Methods and Uses

The macromolecules and pharmaceutically acceptable salts thereof find use in methods of preventing or reducing the likelihood of transmission of Zika virus, and in preventing or reducing the likelihood of diseases, disorders or symptoms associated with a Zika virus infection. The virus is classified as: Group IV, positive sense, single-stranded RNA virus; Family—Flaviviridae; Genus—Flavivirus; Species: Zika virus.

Examples of diseases and disorders include Zika virus disease, Zika fever, and Guillain-Barré syndrome. Symptoms of Zika virus disease include fever, rash and joint pain. A further example of a disorder associated with a Zika virus infection is a fetal birth defect, for example a brain defect such as microcephaly.

Zika virus can be transmitted from one individual to another through sexual intercourse. Typically the individual is a human individual. The virus can be found in semen, as well as in blood. It is envisaged that the macromolecules of the present invention find use in preventing or reducing the likelihood of Zika virus transmission during sexual intercourse, and in preventing the spread of conditions associated with a Zika virus infection. In one embodiment the compounds of the present disclosure are topical antiviral agents intended for application to the vaginal or rectal mucosa. In one embodiment the compounds of the present disclosure are topical antiviral agents intended for application to the penis. For example, a composition comprising the macromolecule (or salt thereof), such as a lubricant composition for sexual intercourse, may be topically administered to the vagina prior to sexual intercourse. As a further example, a condom coated with a composition comprising the macromolecule (or salt thereof) may be used during sexual intercourse. In some embodiments, the macromolecule (or salt thereof) is used for preventing or reducing the likelihood of transmission of Zika virus to a female individual. In some embodiments, the macromolecule is used for preventing or reducing the likelihood of a disease, disorder or symptom associated with a Zika virus infection in a female individual.

It is further contemplated that the macromolecules may find use in additional applications, for example in handwash or antimicrobial compositions, or in mosquito repellent compositions for preventing or reducing the likelihood of transmission of Zika virus.

### Compositions and Delivery Systems

In some embodiments, a composition comprising the macromolecule or a pharmaceutically acceptable salt thereof and a pharmaceutically acceptable carrier is used. Such compositions are contemplated as typically being for topical administration. The formulation of such compositions is well known to persons skilled in this field. The carrier, excipient or diluent may include one or more of any and all conventional solvents, dispersion media, fillers, solid carriers, aqueous solutions, coatings, viscosity modifying agents, isotonic agents, and absorption enhancing or delaying agents, activity enhancing or delaying agents and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional carrier andlor diluent is incompatible with the active ingredient, use thereof in the compositions of the present invention is contemplated.

Vehicles suitable for topical administration include oil-in-water and water-in-oil emulsions, white petrolatum, hydrophilic petrolatum, lanolin emulsions, polyethylene glycols, cocoa butter, buffering agents (including Carbopol 971P), emollient oils (e.g. water-soluble oils including, for example, polyethylene glycol), a lubricating gel (including, for example, water, propylene glycol, hydroxyethyl cellulose, benzoic acid and sodium hydroxide), a water-soluble oil (including, for example, glycerine, propylene glycol, polyquaternium #5, methyl paraben and propyl paraben), a cream (including, for example. benzyl alcohol, cetearyl alcohol, cetyl esters, wax, octyldodecanol, polysorbate 60, purified water, and sorbitan monostearate), and the like. Preferably the composition comprises ingredients selected from the group consisting of sodium hydroxide, water soluble oils, buffering agents, propylene glycol, glycerine, methyl paraben, propyl paraben, polyethylene glycol, edetate disodium dehydrate and water;

Preferably, the carriers, excipients and/or diluents include one or more of the group consisting of sodium hydroxide, water soluble oils, buffering agents, propylene glycol, glycerine and water. More preferably, the carriers, excipients and/or diluents include sodium hydroxide, edetate disodium dihydrate, methyl paraben, propyl paraben, Carbopol 971P, propylene glycol, glycerine, and purified water in combination.

In some embodiments compositions may be formulated in dosage unit form for ease of administration and uniformity of dosage. Dosage unit form as used herein refers to physically discrete units suited as unitary dosages for the individuals to be treated; each unit containing a predetermined quantity of active ingredient calculated to produce the desired prophylactic effect in association with the required pharmaceutical carrier and/or diluent.

A variety of topical administration routes are available. The methods of this disclosure, generally speaking, may be practices using any mode of administration that is medically acceptable, meaning any mode that provides prophylactic or therapeutic levels of the active component without causing clinically unacceptable adverse effects. Such modes of administration include the vaginal, rectal, and dermal routes. Suitable formulations for topical, particularly vaginal or rectal, administration include solutions, suspensions, gels, lotions, foams, films, jellies, and creams. In some embodiments, the macromolecule is administered in the form of a vaginal gel or lubricant.

For example, the macromolecule (or salt thereof) may be present in the form of a gel formulation. In a gel formulation, a rheology modifier may for example be present, in an amount of 2-10%, especially about 5%. Example of gel-forming substances may include natural and synthetic polymers. Gel formulations of the macromolecules may for example include a chelating agent, such as a polyaminocarboxylic acid. A particularly useful chelating agent is ethylenediamine tetraacetic acid (EDTA) and its salts. Other ingredients that may be included in the gel formulation include preservatives such as parabens, for example methylparaben and propylparaben or mixtures thereof, solvents such as water, pH adjusting agents such as hydroxide and emollients and humectants such as glycerine and propylene glycol.

By way of further example, the macromolecule (or salt thereof may) be present in the form of a lubricant formulation. Ingredients that may be included in the lubricant formulation include preservatives such as parabcns, for example methylparaben and propylparaben or mixtures thereof, solvents such as water, pH adjusting agents such as hydroxide and emollients and humectants such as glycerine and/or propylene glycol. . A further component may be hydroxyethylcellulose.

Depending on the intended mode of administration, the compositions may comprise a mucoadhesive polymer. Examples of mucoadhesive polymers include poly(acrylates), chitosan, cellulose derivatives, hyaluronic acid derivatives, pectin, traganth, starch, poly(ethylene glycol), sulfated polysaccharides, carrageenan, sodium alginate and gelatine.

The present disclosure also contemplates the use of an antiviral delivery system comprising:

a) a prophylactic device; and

b) a composition comprising the macromolecule or a pharmaceutically acceptable salt thereof and a pharmaceutically acceptable carrier.

The prophylactic device may be of any suitable type. For example, a condom, a cervical cap, contraceptive diaphragm, vaginal sponge, or the like may be used. A condom is preferred.

The composition is typically selected so as to be compatible with the prophylactic device (i.e. does not alter the integrity of the prophylactic device). The composition is also typically selected so as to be suitable for topical administration, e.g. compatible with sexual organs and/or mucous membranes, such as vaginal and/or rectal membranes, physiological pH.

The composition preferably has a viscosity such that it remains in contact with the prophylactic device for an extended period of time, and does not flow off the prophylactic device on contact.

The composition may be carried on the prophylactic device in any suitable manner. Examples include, but are not limited to, the composition being carried on a surface of the prophylactic device (including the internal surface, the external surface or both surfaces of the device), impregnated into the prophylactic device, covalently bound to a surface of the prophylactic device, and the like. Preferably, the composition covers at least a substantial portion of the external surface and/or the internal surface of the prophylactic device (e.g. condom).

When used in conjunction with or as part of a prophylactic device, the composition comprising the macromolecule or salt thereof may be provided, for example, in the form of a solution, suspension, lotion, jelly, foam, gel or cream, and may contain carriers, excipients and/or diluents as discussed above in relation to compositions. The composition is formulated to be suitable for use with the material of the prophylactic device.

In some embodiments a condom coated with a composition comprising SPL7013 and a pharmaceutically acceptable carrier is used.

### Dosages

The methods of the present disclosure require administration of an effective amount of macromolecule or pharmaceutically acceptable salt thereof, or of compositions comprising the macromolecule. An “effective amount” means an amount necessary to at least partially attain the desired response, or to delay the onset of, inhibit the progression of or halt altogether infection. An effective amount for a human patient may, for example, fall within the range of about 0.5 mg to about 5 mg per dosage.

When used in a composition, the macromolecule or salt thereof may for example be present in the composition in an amount of from about 0.1% w/w to about 10% w/w of the composition, or from about 0.2% w/w/ to about 5% w/w, or about 0.5% w/w of the composition.

In some embodiments, the dosage may be administered in a volume of 0.1 to 50 ml composition, especially 0.2 ml to 1 ml, especially 1 to 25 ml and more especially about 5 ml.

When used in a delivery system (e.g. as a condom coated with a composition comprising the macromolecule or salt thereof), the macromolecule or salt thereof may for example be present in the composition in an amount of from about 0.1% w/w to about 10% w/w of the composition, or from about 0.2%w/w/ to about 5% w/w, or about 0.5% w/w of the composition.

When used in a delivery system (e.g. as a condom coated with a composition comprising the macromolecule or salt thereof), the amount of antiviral composition included in the delivery system according to the present disclosure may for example be from about 0.10 g to about 2 g, or from about 0.1 g to about 0.5 g, or from about 0.1 g to about 0.25 g.

The macromolecule may be administered on a dosage regimen that provides the desired effect. For example, the macromolecule or salt thereof, or composition comprising the macromolecule or salt, may be administered from 1 to 6 times per day, from 1 to 4 times per day, or from 1 to 3 times per day. In some embodiments a composition comprising the macromolecule or salt thereof is administered prior to or concomitant with sexual intercourse.

### Coadministration

Whilst in some embodiments of the present disclosure, the macromolecules or salts thereof may be the sole active ingredients used, in other embodiments the macromolecule is used arc used in combination with one or more further active ingredients, e.g. a further active ingredient for preventing microbial infections. For example, the macromolecules may be used in combination with an antiviral, antifungal, anti-parasitic, antibacterial agent and/or contraceptive agent. Examples of further active agents include podophyllin, tetracycline, nyastatin, fluconazole, metronidazole, acyclovir, penicillin, cefotazime, spectinomycin, retrovir, erythromycin, ceftriaxone, cotrimoxazole, benzyl benzoate, malathion, nonoxynol-9, octoxynol-9, menfegol, progestin, estrogen, and estradiol. The macromolecules or salts thereof may also be used in combination with nonsteroidal anti-inflammatory drug (NSAID). For example, the NSAID may be used to treat the symptoms of a Zika virus infection, whilst the macromolecule or salt thereof may be used to prevent transmission of the virus to another individual.

The present invention will now be more fully described with reference to the accompanying examples. It should be understood, however, that the description following is illustrative only and should not be taken in any way as a restriction on the generality of the invention described above.

### EXAMPLES

### Example 1

### Anti-Zika Virus Cytoprotection Assay:

### Assay Methodology

Inhibition of virus-induced cytopathic effects (CPE) and cell viability following Zika virus replication in Vero cells was measured by XTT tetrazolium dye. Cells were seeded in 96-well flat-bottom tissue culture plates and allowed to adhere overnight.

Following overnight incubation, SPL7013 or the ribavirin control were added to the plate. Following approximately 10 minutes incubation at room temperature, virus diluted to a pre-determined titer to yield 85 to 95% cell killing at 4 days post-infection was added to the plate. The plate was incubated at 37° C., 5% CO₂ for four days and cell viability was measured by XTT staining. The optical density of the cell culture plate was determined spectrophotometrically at 450 and 650. Percent CPE reduction of the virus-infected wells and the percent cell viability of uninfected drug control wells were calculated to determine the EC₅₀ and TC₅₀ values using four parameter curve fit analysis.

### Cell Preparation

Vero cells (African green monkey kidney cells) were passaged in DMEM supplemented with 10% FBS, 2 mM L-glutamine, 100 U/mL penicillin, and 100 μg/mL streptomycin in T-75 flasks. The cells were resuspended at 5×10³ cells per well in tissue culture medium and added to flat bottom microtiter plates in a volume of 100 μL.

The plates were incubated at 37° C./5% CO₂ overnight to allow for cell adherence. Monolayers were observed to be approximately 70% confluent.

### Virus Preparation

Zika virus obtained from ZeptoMetrix (catalog # 0810092CF; Buffalo, N.Y.) was titrated in Vero cells to define the inoculum for use in the Zika virus antiviral assay. Virus was diluted into assay medium (DMEM supplemented with 2% heat-inactivated FBS, 2 mM L-glutamine, 100 U/mL penicillin, and 100 μg/mL streptomycin) such that the amount of virus added to each well in a volume of 100 μL was the amount determined to yield 85 to 95% cell killing at 4 days post-infection.

### Plate Format

Each plate contained cell control wells (cells only), virus control wells (cells plus virus), triplicate drug toxicity wells per compound (cells plus drug only), as well as triplicate experimental wells (drug plus cells plus virus).

### Efficacy and Toxicity XTT

Following incubation at 37° C. in a 5% CO₂ incubator for four days, the test plates were stained with the tetrazolium dye XTT (2,3-bis(2-methoxy-4-nitro-5-sulfophenyl)-5-[(phenylamino)carbonyl]-2H-tetrazolium hydroxide). XTT/PMS stock was prepared immediately before use by adding 40 μL of PMS (Phenazine methosulfate (PMS) solution at 0.15 mg/mL in PBS) per ml of XTT solution (1 mg/mL in RPMI1640). Fifty microliters of XTT/PMS was added to each well of the plate and the plate was reincubated for 4 hours at 37° C. Plates were sealed and mixed and the plate was read spectrophotometrically at 450/650 nm with a Molecular Devices Vmax plate reader.

### Data Analysis

The raw data was analyzed using four parameter curve fit calculations.

### Results

The results are shown in the table 1 below:

| | Vero/Zika Virus_{ZM01} | | |
|---|---|---|---|
| | EC₅₀ | TC₅₀ | Therapeutic |
| Compound | (μg/mL) | (μg/mL) | Index |
| Ribavirin | 1.13 | 57.3 | 50.7 |
| | 4.13 | 57.1 | 13.8 |
| SPL7013 | <20.0 | 640 | >32.0 |
| | 0.86 | >100 | >116 |

The experiment was conducted twice, at different concentrations of SPL7013 (20 to 5000 μg/mL, and 0.32 to 100 μg/mL) in order to establish both the EC₅₀ and TC₅₀ values for the compound.

Testing showed that SPL7013 has prophylactic activity against Zika virus by the measurement of virus-induced cytopathic effect (CPE) inhibition and comparison to the positive control ribavirin. The EC50 for SPL7013 was calculated as 0.86 μg/mL; (the positive control was 1.13 μg/mL and 4.13 μg/mL). The TC50 value for SPL7013 was 640 μg/mL (positive control ˜57 μg/mL) resulting in a calculated therapeutic index of 744 for SPL7013 (ribavirin 50.7 and 13.8).

### Comparative Example 1

### Inactivity of Dendrimeric Compounds Against Dengue Virus and West Nile Fever Virus

The activity of dendrimeric compounds against Dengue virus (D) and West Nile Fever virus (WNF) was tested.

a) The compound [BHAlys][Lys]16[CONH-Ph-3,5-(SO3Na)2]32 (SPL compound 7674) was tested for activity against West Nile Fever virus (DSP0114) (the compound has a sulfonic acid- or sulfonate-containing moiety which is

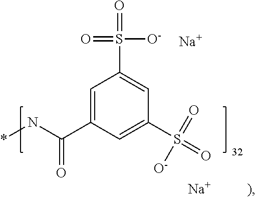

A 4 concentration assay was used. The positive control was interferon-alpha. The results were as follows:
EC50VIS: 22 ug/mL
CC50 VIS: >100 ug/mL
EC50 NR: 34 ug/mL
CC50 NR: >100 ug/mL

The compound was not active.

b) The compound [BHAlys] [Lys]16 [CSNH-4-Ph(SO3Na)]32 (SPL compound 2792) was tested for activity against Dengue Type 2 virus (DSP0196). (the compound has a sulfonic acid- or sulfonate-containing moiety which is

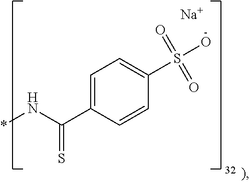

The results were as follows:
EC50 VIS: >100 ug/mL
CC50 VIS: 31.2 ug/mL
EC50 NR: 50.3 ug/mL
CC50 NR: 50.1 ug/mL

The compound was not active.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

This application claims priority from Australian Provisional Application No. 2016901650 entitled “Method of prophylaxis of zika virus infection” filed on 5 May 2016. The entire contents of that application are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The steps, features, integers, compositions and/or compounds disclosed herein or indicated in the specification of this application individually or collectively, and any and all combinations of two or more of said steps or features.

1. A method of preventing or reducing the likelihood of transmission of Zika virus during sexual intercourse to a first individual from a second individual infected with Zika virus, comprising:
topically administering to the first and/or second individual an effective amount of a macromolecule or a pharmaceutically acceptable salt thereof, wherein the macromolecule comprises a dendrimer of 1 to 8 generations with one or more sulfonic acid- or sulfonate-containing moieties attached to one or more surface groups of the dendrimer.

2. A method of preventing or reducing the likelihood of a disease, disorder or symptom associated with a Zika virus infection in a first individual, by preventing transmission of Zika virus during sexual intercourse to the first individual from a second individual infected with Zika virus, the method comprising:
topically administering to the first and/or second individual an effective amount of a macromolecule or a pharmaceutically acceptable salt thereof, wherein the macromolecule comprises a dendrimer of 1 to 8 generations with one or more sulfonic acid- or sulfonate-containing moieties attached to one or more surface groups of the dendrimer.

3. A method as claimed in claim 1, wherein the macromolecule or pharmaceutically acceptable salt thereof is a dendrimer comprising lysine building units of from 3 to 6 generations, and the sulfonic acid- or sulfonate-containing moieties are napthyldisulfonate moieties.

4. A method as claimed in claim 3, wherein the macromolecule is astrodrimer.

5. A method as claimed in claim 1, wherein the macromolecule is present in a lubricant composition for sexual intercourse.

6. A method as claimed in claim 1, which comprises using during sexual intercourse a system comprising:
a) a prophylactic device; and
b) the macromolecule or a pharmaceutically acceptable salt thereof.

7. A method as claimed in claim 6, wherein the system is a condom coated with a composition comprising the macromolecule or a pharmaceutically acceptable salt thereof and a pharmaceutically acceptable carrier.

8. A method as claimed in claim 2, wherein the disease, disorder or symptom is Zika virus disease.

9. A method of preventing or reducing the likelihood of transmission of Zika virus to an individual, comprising:
topically administering to the individual an effective amount of a macromolecule or a pharmaceutically acceptable salt thereof, wherein the macromolecule comprises a dendrimer of 1 to 8 generations with one or more sulfonic acid- or sulfonate-containing moieties attached to one or more surface groups of the dendrimer.

10. (canceled)

11. A method as claimed in claim 9, wherein the macromolecule or pharmaceutically acceptable salt thereof is a dendrimer comprising lysine building units of from 3 to 6 generations, and the sulfonic acid- or sulfonate-containing moieties are napthyldisulfonate moieties.

12. A method as claimed in claim 11, wherein the macromolecule is astrodimer.

13-14. (canceled)

15. A method as claimed in claim 9, wherein the macromolecule is present in a lubricant composition for sexual intercourse.

16. A method as claimed in claim 2, wherein the macromolecule or pharmaceutically acceptable salt thereof is a dendrimer comprising lysine building units of from 3 to 6 generations, and the sulfonic acid- or sulfonate-containing moieties are napthyldisulfonate moieties.

17. A method as claimed in claim 16, wherein the macromolecule is astodrimer.

18. A method as claimed in claim 2, wherein the macromolecule is present in a lubricant composition for sexual intercourse.

19. A method as claimed in claim 2, which comprises using during sexual intercourse a system comprising:
a) a prophylactic device; and
b) the macromolecule or a pharmaceutically acceptable salt thereof.

20. A method as claimed in claim 19, wherein the system is a condom coated with a composition comprising the macromolecule or a pharmaceutically acceptable salt thereof and a pharmaceutically acceptable carrier.
